(19) United States
(12) Patent Application Publication
(10) Pub. No.: US 2011/0270415 A1
(43) Pub. Date: Nov. 3, 2011

(54) ARTIFICIAL KNEE JOINT

(76) Inventors: Chien-Wen Chen, Taipei County (TW); Chien-Cheng Chen, Taipei County (TW)

(21) Appl. No.: 12/770,015

(22) Filed: Apr. 29, 2010

(51) Int. Cl. A61F 2/64

(52) U.S. Cl. 623/44

(57) ABSTRACT

An artificial knee joint includes a lower joint member, an axle, a kneecap member, and a press member. The lower joint member is formed with a horizontally extending axle-receiving hole, and a clearance extending from and in spatial communication with the axle-receiving hole. The axle is received in the axle-receiving hole. The kneecap member is pivoted to the lower joint member, and is adapted to be connected to a prosthetic thigh. The press member is mounted to the kneecap member for pressing the lower joint member when the kneecap member pivots in a direction relative to the lower joint member. Therefore, the clearance and the axle-receiving hole are constricted to result in tightened connection between the lower joint member and the axle and resistance to relative rotation between the lower joint member and the axle.

### BACKGROUND OF THE INVENTION

1. Field of the Invention

The present invention relates to an artificial knee joint, more particularly to an artificial knee joint capable of providing resistance to relative rotation between a lower joint member and an axle thereof.

2. Description of the Related Art

In a prosthesis for a femur amputee, an artificial knee joint for connecting a prosthetic thigh to a prosthetic lower leg is particularly important. When a prosthesis wearer walks on an uneven surface and wants to stop walking, the prosthesis wearer will fall down if the artificial knee joint is unable to resist rotation.

Taiwanese Utility Model No. M284363 discloses an artificial knee joint including a mechanism for preventing the prosthesis wearer from falling down. The disclosed artificial knee joint includes an axle received in an axle-receiving hole that has an inner wall formed with a plurality of grooves parallel to the axle. Each of the grooves has a narrow portion, and receives a rod. When the rods move into the narrow portions, the rods are brought into tight contact with the axle and resist rotation of the axle. Therefore, the prosthesis wearer will not fall down when the prosthesis wearer stops walking. However, the structure of the disclosed artificial knee joint is complicated. Further, the disclosed artificial knee joint is unable to adjust sensitivity of resistance to rotation of the axle, and is not configured to suit the individual weight of different prosthesis wearers.

### SUMMARY OF THE INVENTION

Therefore, an object of the present invention is to provide an artificial knee joint capable of providing resistance to relative rotation between a lower joint member and an axle thereof.

Another object of the present invention is to provide an artificial knee joint capable of adjusting sensitivity of resistance to relative rotation between a lower joint member and an axle thereof.

A further object of the present invention is to provide an artificial knee joint that can suit the individual weight of different prosthesis wearers.

According to the present invention, an artificial knee joint is adapted for connecting a prosthetic thigh to a prosthetic lower leg. The artificial knee joint comprises a lower joint member, an axle, a biasing assembly, a kneecap member, and a press member.

The lower joint member includes a base portion, a hole-defining portion, and a lever portion. The hole-defining portion has a first end connected to the base portion, and a second end. The hole-defining portion cooperates with the base portion to define a horizontally extending axle-receiving hole. The lever portion extends from the second end of the hole-defining portion and extends above the base portion. The axle is received in the axle-receiving hole. The biasing assembly is mounted to the base portion of the lower joint member, and is disposed to abut against the lever portion of the lower joint member for providing a biasing force to the lever portion. A clearance extending from and in spatial communication with the axle-receiving hole is formed between the lever portion and the base portion. The kneecap member is pivoted to the lower joint member, and is adapted to be connected to the prosthetic thigh.

The press member is mounted to the kneecap member for pressing the lever portion to move toward the base portion against biasing action of the biasing assembly when the kneecap member pivots in a direction relative to the lower joint member. Movement of the lever portion toward the base portion constricts the clearance and the axle-receiving hole to result in tightened connection between the lower joint member and the axle and resistance to relative rotation between the lower joint member and the axle.

### BRIEF DESCRIPTION OF THE DRAWINGS

Other features and advantages of the present invention will become apparent in the following detailed description of the preferred embodiment with reference to the accompanying drawings, of which:

FIG. 1 is a fragmentary exploded perspective view of a preferred embodiment of an artificial knee joint according to the present invention for illustrating assembly relationships among components of the artificial knee joint;

FIG. 2 is a cross-sectional view of the artificial knee joint;

FIG. 3 is a cross-sectional view of the artificial knee joint for illustrating resistance to relative rotation between a lower joint member and a first axle of the artificial knee joint; and

FIG. 4 is a fragmentary cross-sectional view illustrating a buffer assembly of the artificial knee joint.

### DETAILED DESCRIPTION OF THE PREFERRED EMBODIMENT

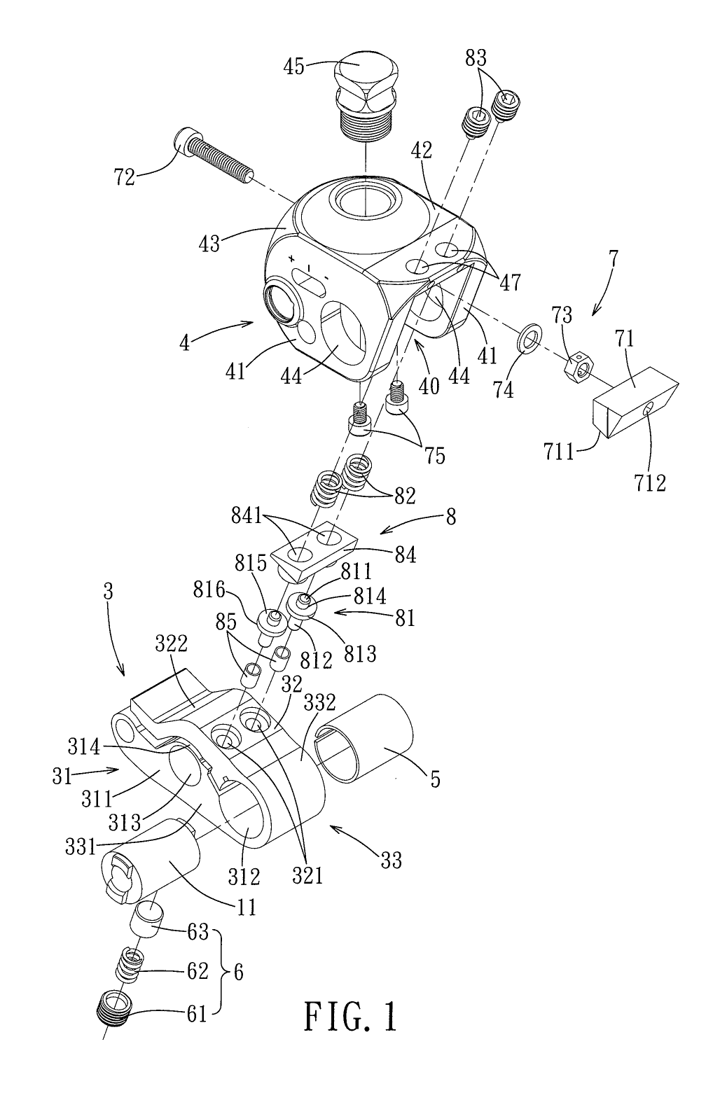

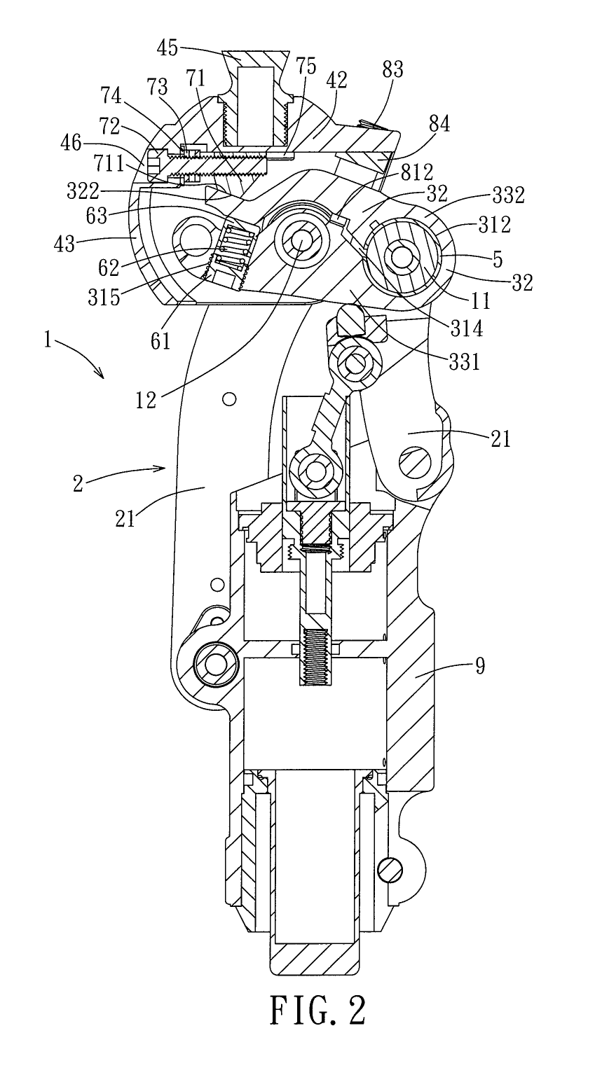

Referring to FIGS. 1 and 2, the preferred embodiment of an artificial knee joint 1 of the present invention is adapted for connecting a prosthetic thigh (not shown) to a prosthetic lower leg (not shown). The artificial knee joint 1 includes a link assembly 2, a lower joint member 3, a kneecap member 4, a C-shaped sleeve 5, a biasing assembly 6, a press member 7, and a lower seat 9.

The link assembly 2 includes four links 21, but only two of the links 21 are visible in FIG. 2 due to the angle of view. The kneecap member 4 is adapted to be connected to the prosthetic thigh, and is disposed above and pivoted to the lower joint member 3. The lower seat is disposed under the lower joint member 3 for connection of the prosthetic lower leg, and is connected to the lower joint member 3 by the links 21 of the link assembly 2. The sleeve 5 and the biasing assembly 6 are disposed within the lower joint member 3, and the press member 7 is mounted to the kneecap member 4 and between the lower joint member 3 and the kneecap member 4. Moreover, the artificial knee joint 1 further includes a first axle 11 and a second axle 12 that are received in the lower joint member 3 and that are connected to the links 21 of the link assembly 2. It should be noted that, in the structure of the link assembly 2, the entire link assembly 2 is prevented from movement when one of the first and second axles 11, 12 is locked.

The lower joint member 3 includes a base portion 31, a lever portion 32, and a hole-defining portion 33 having a first end 331 and a second end 332. The first end 331 of the hole-defining portion 33 is connected to the base portion 31, and the lever portion 32 extends from the second end 332 of the hole-defining portion 33 and extends above the base portion 31. The hole-defining portion 33 cooperates with the base portion 31 to define a horizontally extending first axle-receiving hole 312 for receiving the first axle 11 so as to allow relative rotation between the first axle 11 and the lower joint member 3. A clearance 314 extending from and in spatial communication with the first axle-receiving hole 312 is formed between the lever portion 32 and the base portion 31. The sleeve 5 is sleeved on the first axle 11, and is disposed in the first axle-receiving hole 312.

The base portion 31 of the lower joint member 3 has an opposing pair of lateral sides 311, and is formed with a second axle-receiving hole 313 through the lateral sides 311 for receiving the second axle 12. The base portion 31 is further formed with a threaded accommodating hole 315 remote from the first axle-receiving hole 312 for containing the biasing assembly 6 therein. The lever portion 32 of the lower joint member 3 has an inclined to-be-pressed surface 322, and is formed with a pair of first through holes 321 that extend vertically through the lever portion 32 and that are horizontally spaced apart from each other.

The kneecap member 4 includes an opposing pair of vertical plate parts 41 spaced apart from each other, a horizontal plate part 42 disposed above and connected to the vertical plate parts 41, and a front plate part 43 disposed in front of and connected to the vertical and horizontal plate parts 41, 42. Each of the vertical plate parts 41 is formed with a lateral through hole 44. The vertical, horizontal and front plate parts 41-43 cooperate with and are connected to each other to form an accommodating space 40 for containing the lower joint member 3 in a manner that each of the vertical plate parts 41 is disposed adjacent to one of the lateral sides 311 of the base portion 31 and the lateral through holes 44 are aligned with the second axle-receiving hole 313. The second axle 12 extends through the second axle-receiving hole 313 and the lateral through holes 44 of the vertical plate parts 41 to interconnect pivotally the kneecap member 4 and the base portion 31 of the lower joint member 3. Therefore, the kneecap member 4 is pivoted to the lower joint member 3.

The kneecap member 4 further includes a tetragonal post 45 that is disposed on the top of the horizontal plate part 42 and that is adapted to be connected to the prosthetic thigh. Moreover, the front plate part 43 of the kneecap member 4 is formed with a front through hole 46. The horizontal plate part 42 of the kneecap member 4 is formed with a pair of second through holes 47 that are horizontally spaced apart from each other and that are aligned respectively with the first through holes 321 on the lever portion 32 of the lower joint member 3.

The biasing assembly 6 includes a stop 61 fastened in the accommodating hole 315 of the base portion 31, a restoring spring 62 disposed on the stop 61, and a cap 63 covering the restoring spring 62. The cap 63 projects outwardly of the accommodating hole 315 and reaches the clearance 314 for abutting against the lever portion 32 of the lower joint member 3 such that the restoring spring 62 provides a biasing force to the lever portion 32. It should be noted that the clearance 314 between the lever portion 32 and the base portion 31 can be formed attributed to the biasing force or the designed structure of the lower joint member 3. In this embodiment, the stop 61 is a set screw threadedly engaging the threaded accommodating hole 315.

The press member 7 is mounted to the kneecap member 4 and includes a press block 71, an adjusting component 72, a nut 73, and a washer 74. The press block 71 is disposed above the lever portion 32 of the lower joint member 3, and is non-rotatably disposed in the kneecap member 4. The press block 71 has an inclined contacting surface 711 in contact with the to-be-pressed surface 322 of the lever portion 32, and is formed with a screw hole 712. The adjusting component 72 extends horizontally through the front through hole 46 in the front plate part 43 of the kneecap member 4, is connected to the press block 71, and is operable to move the press block 71 along the to-be-pressed surface 322 for adjusting the clearance 314 between the lever portion 32 and the base portion 31. In this embodiment, the adjusting component 72 is a screw (such as a hexagonal socket screw) threadedly engaging the screw hole 712, and the nut 73 and washer 74 are fixed in the kneecap member 4 and engage the adjusting component 72. Therefore, the adjusting component 72 can be rotated to move the press block 71 along the to-be-pressed surface 322. The press member 7 further includes a stop component 75 disposed below the horizontal plate part for preventing the adjusting component 72 from rearward movement. In this embodiment, the stop component 75 is disposed on a bottom surface of the horizontal plate part 42 by threaded engagement.

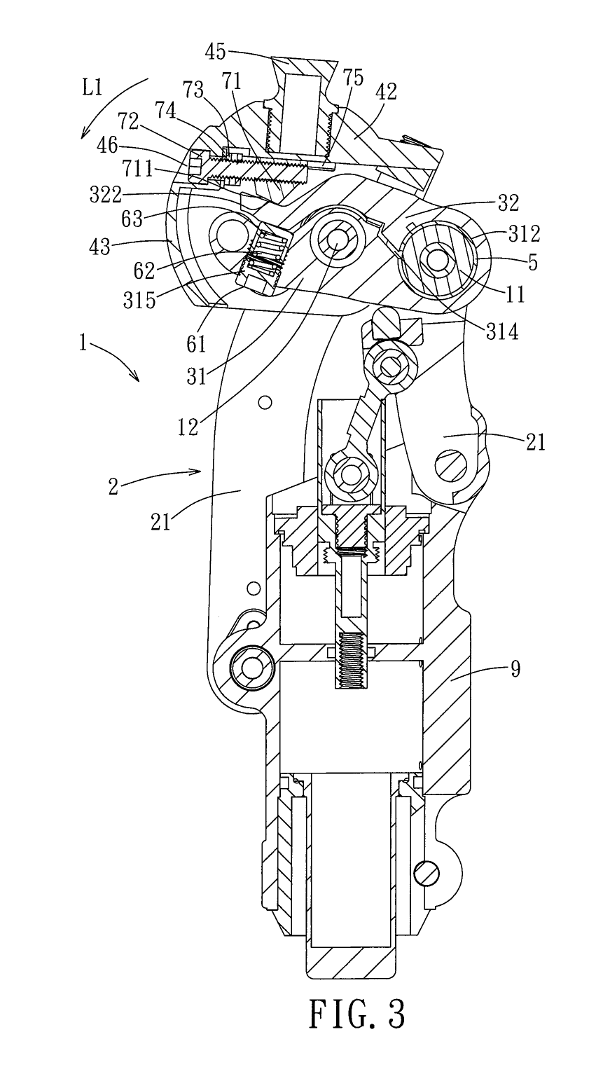

When a prosthesis wearer wearing the artificial knee joint 1 wants to stop walking, the kneecap member 4 will pivot in a direction L1 relative to the lower joint member 3 as shown in FIG. 3. At this time, the press block 71 of the press member 7 moves with the kneecap member 4 for pressing the lever portion 32 to move toward the base portion 31 against biasing action of the biasing assembly 6. Movement of the lever portion 32 toward the base portion 31 constricts the clearance 314 and the first axle-receiving hole 312 to result in tight gripping of the first axle 11 by the sleeve 5 and resistance to relative rotation between the lower joint member 3 and the first axle 11. Therefore, the first axle 11 is locked so as to prevent the prosthesis wearer from falling down.

Moreover, when the press block 71 presses the lever portion 32 to move toward the base portion 31 against the biasing action of the biasing assembly 6, the cap 63 compresses the restoring spring 62 such that the restoring spring 62 deforms and stores a restoring force. When the prosthesis wearer stops walking, the restoring spring 62 restores the lever portion 32, that is to say, the clearance 314 and the first axle-receiving hole 312 are restored to their non-constricted states to once again permit the relative rotation between the lower joint member 3 and the first axle 11.

It should be noted that the size of the clearance 314 affects a sufficient amount of the force to which the lever portion 32 is subjected to result in resistance to the relative rotation between the lower joint member 3 and the first axle 11. The size of the clearance 314 can be adjusted by operating the adjusting component 72 to move the press block 71 along the to-be-pressed surface 322 of the lever portion 32. Namely, sensitivity of the resistance to the relative rotation between the lower joint member 3 and the first axle 11 is adjustable to meet different requirements.

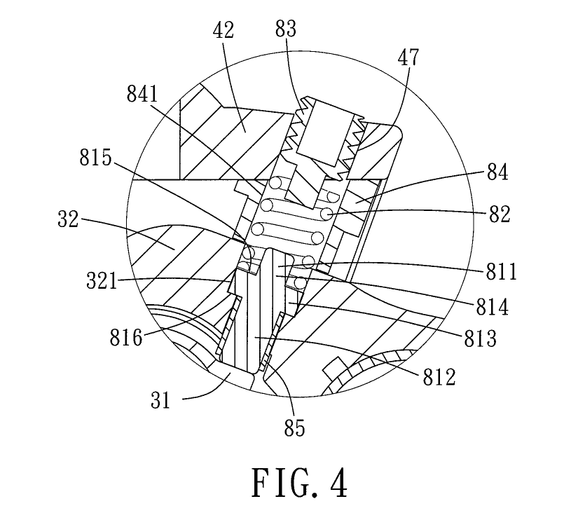

Referring to FIGS. 1, 2 and 4, the artificial knee joint 1 preferably includes a buffer assembly 8 disposed between the kneecap member 4 and the lower joint member 3. The buffer assembly 8 includes a pair of abutting components 81 extending through the lever portion 32 and abutting against the base portion 31, a pair of buffer components 82 disposed between the abutting components 81 and the horizontal plate part 42 of the kneecap member 4, a pair of force-adjusting components 83 each of which is mounted adjustably in one of the second through holes 47 in the horizontal plate part 42 and abuts against one of the buffer components 82, and a containing component 84 within which the buffer components 82 are disposed. In this embodiment, the buffer components 82 are compression springs and the force-adjusting components 83 are screws threadedly engaging the second through holes 47, respectively.

Each of the abutting components 81 includes a first post segment 811 having a bottom edge 814, a flange 813 projecting outwardly and radially from the bottom edge 814 of the first post segment 811 and having a top surface 815 and a bottom surface 816, and a second post segment 812 projecting downwardly from the bottom surface 816 of the flange 813 and abutting against the base portion 31. Each of the buffer components 82 is sleeved on the first post segment 811, and abuts against the top surface 815 of the flange 813. By virtue of interconnections among the abutting components 81, the buffer components 82 and the force-adjusting components 83, the buffer assembly 8 provides an upward supporting force to the kneecap member 4 for supporting the weight of the prosthesis wearer. Moreover, for different prosthesis wearers with different weights, the force-adjusting components 83 are operable to compress or release the buffer components 82 to thereby adjust spring forces of the buffer components 82. The containing component 84 is a rubber block and is formed with a pair of containing holes 841 for containing the buffer components 82 therein, respectively. By virtue of the containing component 84, the interconnections among the abutting components 81, the buffer components 82 and the force-adjusting components 83 are relatively stable. The buffer assembly 8 further includes a pair of friction-reducing sleeves 85 sleeved respectively on the second post segments 812 for reducing friction between the base portion 31 and the second post segments 812.

In summary, when the lever portion 32 of the lower joint member 3 is pressed, constricting of the first axle-receiving hole 312 results in tight gripping of the first axle 11 by the sleeve 5 to resist the relative rotation between the lower joint member 3 and the first axle 11. Therefore, the link assembly 2 is locked to thereby prevent the prosthesis wearer from falling down. Moreover, the press member 7 is operable to adjust the sensitivity of the resistance to the relative rotation between the lower joint member 3 and the first axle 11, and the buffer assembly 8 is operable to adjust the supporting force.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. An artificial knee joint adapted for connecting a prosthetic thigh to a prosthetic lower leg, said artificial knee joint comprising:
a lower joint member including
a base portion,
a hole-defining portion having a first end connected to said base portion, and a second end, said hole-defining portion cooperating with said base portion to define a horizontally extending first axle-receiving hole, and
a lever portion extending from said second end of said hole-defining portion and extending above said base portion;
a first axle received in said first axle-receiving hole;
a biasing assembly mounted to said base portion of said lower joint member and disposed to abut against said lever portion of said lower joint member for providing a biasing force to said lever portion, wherein a clearance extending from and in spatial communication with said first axle-receiving hole is formed between said lever portion and said base portion;
a kneecap member pivoted to said lower joint member and adapted to be connected to the prosthetic thigh; and
a press member mounted to said kneecap member for pressing said lever portion to move toward said base portion against biasing action of said biasing assembly when said kneecap member pivots in a direction relative to said lower joint member, movement of said lever portion toward said base portion constricting said clearance and said first axle-receiving hole to result in tightened connection between said lower joint member and said first axle and resistance to relative rotation between said lower joint member and said first axle.

2. The artificial knee joint as claimed in claim 1, further comprising a C-shaped sleeve sleeved on said first axle and disposed in said first axle-receiving hole,
wherein constricting of said first axle-receiving hole results in tight gripping of said first axle by said sleeve to resist relative rotation between said lower joint member and said first axle.

3. The artificial knee joint as claimed in claim 1, wherein said press member includes a press block that is disposed above said lever portion of said lower joint member and that has a contacting surface, and said lever portion has a to-be-pressed surface in contact with said contacting surface of said press block.

4. The artificial knee joint as claimed in claim 3, wherein said contacting surface and said to-be-pressed surface are inclined surfaces, and said press block is movable along said to-be-pressed surface for adjusting said clearance between said lever portion and said base portion.

5. The artificial knee joint as claimed in claim 4, wherein said press member further includes an adjusting component that extends horizontally through said kneecap member, that is connected to said press block, and that is operable to move said press block along said to-be-pressed surface of said lever portion.

6. The artificial knee joint as claimed in claim 5, wherein said press block is formed with a screw hole and is non-rotatably disposed in said kneecap member, said adjusting component being a screw that threadedly engages said screw hole, said press member further including a nut that is fixed in said kneecap member and that engages said adjusting component.

7. The artificial knee joint as claimed in claim 1, wherein said press member is adjustable relative to said lever portion of said lower joint member for adjusting said clearance between said lever portion and said base portion.

8. The artificial knee joint as claimed in claim 1, further comprising a buffer assembly that includes an abutting component extending through said lever portion and abutting against said base portion, and a buffer component disposed between said abutting component and said kneecap member.

9. The artificial knee joint as claimed in claim 8, wherein said buffer component is a compression spring, and said buffer assembly further includes a force-adjusting component that is mounted adjustably to said kneecap member, that abuts against said buffer component, and that is adjustable to adjust a spring force of said buffer component.

10. The artificial knee joint as claimed in claim 9, wherein said abutting component includes a first post segment having a bottom edge, a flange projecting outwardly and radially from said bottom edge of said first post segment and having a top surface and a bottom surface, and a second post segment projecting downwardly from said bottom surface of said flange and abutting against said base portion, said buffer component being sleeved on said first post segment and abutting against said top surface of said flange, said buffer assembly further including a containing component within which said buffer component is disposed.

11. The artificial knee joint as claimed in claim 1, wherein said base portion of said lower joint member has an opposing pair of lateral sides and is formed with a second axle-receiving hole through said lateral sides, said kneecap member including an opposing pair of vertical plate parts spaced apart from each other, each of said vertical plate parts being disposed adjacent to one of said lateral sides of said base portion and being formed with a lateral through hole, said artificial knee joint further comprising a second axle extending through said second axle-receiving hole and said lateral through holes of said vertical plate parts to interconnect pivotally said kneecap member and said base portion.

12. The artificial knee joint as claimed in claim 1, wherein said base portion of said lower joint member is formed with an accommodating hole remote from said first axle-receiving hole for containing said biasing assembly therein, said biasing assembly including a stop fastened in said accommodating hole, a restoring spring disposed on said stop, and a cap covering said restoring spring and projecting outwardly of said accommodating hole for abutting against said lever portion of said lower joint member.
